(19) United States
(12) Patent Application Publication
(10) Pub. No.: US 2009/0048153 A1
(43) Pub. Date: Feb. 19, 2009

(54) COMPOSITION FOR TREATING SULFUR MUSTARD TOXICITY AND METHODS OF USING SAME

(75) Inventors: Shambhu D. Varma, Ellicott City, MD (US); John Petrali, Churchville, MD (US)

Correspondence Address: DLA PIPER LLP (US);ATTN: PATENT GROUP, 500 8th Street, NW, WASHINGTON, DC, 20004-2131, US

(73) Assignees: UNIVERSITY OF MARYLAND, BALTIMORE, Baltimore, MD (US); The Government of the United States, as represented by the Secretary of the Army, Fort Detrick, MD (US)

(21) Appl. No.: 11/720,283

(22) PCT Filed: Dec. 2, 2005
(86) PCT No.: PCT/US05/43341

§ 371 Date: May 25, 2007

Related U.S. Application Data
(60) Provisional application No. 60/632,834, filed on Dec. 3, 2004.

(51) Int. Cl. A61K 38/28; A61K 31/185; A01N 37/12; A61K 31/405; A61K 31/56; A61K 31/355; A61K 31/7004

(52) U.S. Cl. 514/4; 514/578; 514/23; 514/563; 514/458; 514/420; 514/183

(57) ABSTRACT

One embodiment of the present invention provides a composition, comprising, in amounts effective to treat sulfur mustard or half sulfur mustard induced toxicity or skin damage: an agent that inhibits alkylation of —SH and >NH protein groups; an agent that reduces —SS— to SH; a scavenger of reactive oxygen species; a substrate that maintains tissue reduction-oxidation status; an agent that protects against invading inflammatory cells and associated oxidative stress; an antagonist of prostaglandin synthesis; and an agent that induces tissue regeneration. Methods of using the composition are also provided.

### CROSS REFERENCE TO RELATED APPLICATIONS

This application is based on U.S. Provisional Application Ser. No. 60,632,834, filed Dec. 3, 2004, the entire contents of which are hereby incorporated by reference, the same as if set forth at length.

### STATEMENT REGARDING FEDERALLY SPONSORED RESEARCH

The subject matter herein was supported by the U.S. Army Medical Research Institute for Chemical Defense under grant number DAMD 17-98-1-8361. The U.S. government has certain rights to this invention.

### BACKGROUND OF THE INVENTION

1. Field of the Invention

The present invention relates to the treatment of skin damage caused by exposure to sulfur mustard (mustard gas) and compositions therefor.

2. Discussion of the Background

Sulfur mustard (HD, or “mustard”) is one of the most common chemical warfare agents. Sulfur mustard affects the performance of combat personnel soon after their exposure to it. It is usually dispersed either neat or as an aerosol mist. First used by the German Army in 1917, HD was later used in World War II, the Iran-Iraq conflict, and the Persian Gulf Operation. One early manifestation of exposure to HD consists of a generalized irritation and intense dermal itching all over the body. This is soon followed by erythema, lesions, and blisters in various regions of the skin, loss of vision, and impaired respiration. The lesions are typically more pronounced in the moist areas of the body such as the eyelids and cornea. Skin irritation, other functional disablements, and tissue necrosis can be triggered by exposure to HD levels as low as 1/10,000. Despite its long history of use in combat as a war gas (mustard gas), treatments for its damaging effects remain heretofore unavailable. One reason for the lack of therapy is the post-exposure etiological complexity. Further, the dual solubility of HD increases its breadth of action, ranging from the cell membrane to the cytosol, and the elements contained within (i.e., the mitochondria and the nucleus).

Mechanistically, alkylation of —NH₂ and —SH groups present in tissue structure and soluble constituents of the integral cells is believed to be one of the initial biochemical reactions involved in mustard exposure (1-3). This can take place at sites extending from the outer limiting cell membrane to the cytosolic, mitochondrial and intranuclear components. Alkylation reactions at the cell membrane level damage transport functions by altering the structures of the various channels and pumps required to maintain a selective and precisely controlled intracellular composition. Intra-cellularly, alkylation of various enzymes and consequent change in structure accompanied by alterations in the affinity for respective substrates leads to the inhibition of cellular metabolism and ATP synthesis, which is required to drive the transport pumps and sustain many biosynthetic and repair activities. Inhibition of cellular metabolism in general is known to divert tissue oxygen from its divalent pathway of reduction to water to the monovalent pathway resulting in the generation of super-oxide and other oxyradical species that are highly toxic, inflicting an oxidative stress to the tissue.

### DESCRIPTION OF THE FIGURES

FIG. 1 shows the external appearance of treated and untreated skin after 5 days of Half Mustard (2-chloroethyl-ethyl sulfide, abbreviated as CEES) application.

FIG. 2 shows the histology of normal skin and treated and untreated skin exposed to CEES for a first group of animals.

FIG. 3 shows the histology of normal skin and treated and untreated skin exposed to CEES for a second group of animals.

FIG. 4 shows the histology of normal skin and treated and untreated skin exposed to CEES for a third group of animals.

FIG. 5 shows an assessment of apoptosis by histochemical detection of DNA fragmentation using TUNEL staining for normal skin and treated and untreated skin exposed to CEES.

FIG. 6 shows an assessment of apoptosis by histochemical detection of DNA fragmentation using Hoechst staining for normal skin and treated and untreated skin exposed to CEES.

FIG. 7 presents a graphical sketch of events leading to tissue damage by sulfur mustard.

### DETAILED DESCRIPTION OF SEVERAL EMBODIMENTS

The present inventors have found that the pathological manifestations of mustard on the skin and other exposed tissues can be effectively treated and/or prevented by application of a formulation containing a mixture of compounds selected to inhibit alkylation of —NH₂, >NH, and SH groups of biological molecules, e.g., polypeptides, polynucleotides, sterols, steroids, hormones, bile acids, and the like, scavenge and prevent generation of oxy-radicals, and promote tissue regeneration and repair by providing additional metabolic support. The present inventors have demonstrated the effectiveness of such an approach against ocular and skin damage induced by Half Mustard. Such a mixture is also effective against HD damage to the eye.

Heretofore, the distribution patterns of various reactive constituents inside and outside the cell as well as the kinetic variations in the reactions involved apparently were believed to be so diverse that treatment against mustard induced tissue damage has so far not been found adequately beneficial. One embodiment of the present invention, therefore, provides a composition including a mixture of compounds effective to treat and/or prevent Half Mustard (2-chloroethyl-ethyl sulfide, abbreviated as CEES) induced skin damage, which is also effective against damage by sulfur mustard used in combat. In one embodiment, the compositions include compounds serving the individual and/or collective functions listed below:
(1) Competitively inhibit alkylation at —SH and —NH sites, e.g., of one or more proteins of interest;
(2) Cleave (reduce) —SS— to —SH;
(3) Scavenge reactive oxygen species and minimize oxidative stress;
(4) Provide metabolic and regenerative support by supplying additional substrates and maintaining the status of tissue reduction-oxidation;
(5) Protect against the diverse effects of invading inflammatory cells, including the oxidative stress caused by liberation of oxygen radicals during their activation;
(6) Decrease prostaglandin synthesis; and/or
(7) Aid in tissue regeneration.

One embodiment provides a composition effective for the treatment and/or prevention of sulfur mustard and/or Half Mustard toxicity and/or damage, which includes, in an amount effective to treat or prevent sulfur mustard and/or Half Mustard toxicity and/or damage:

an inhibitor of alkylation of —SH and >NH groups of one or more proteins;

a reducing agent that cleaves —SS— to —SH;

a scavenger of reactive oxygen species thereby minimizing oxidative stress;

a substrate to maintain the status of tissue reduction-oxidation;

an agent that protects against invading inflammatory cells, including the oxidative stress caused by liberation of oxygen radicals during the activation of the inflammatory cells;

an antagonist of prostaglandin synthesis; and

an agent that induces tissue regeneration.

One embodiment provides a method for treating or preventing or preventing and treating sulfur mustard and/or Half Mustard toxicity and/or damage, which includes administering to a subject in need thereof a composition, in an amount effective to treat or prevent sulfur mustard and/or Half Mustard toxicity and/or damage:

an inhibitor of alkylation of —SH and >NH groups of one or more proteins;

a reducing agent that cleaves —SS— to —SH;

a scavenger of reactive oxygen species thereby minimizing oxidative stress;

a substrate to maintain the status of tissue reduction-oxidation;

an agent that protects against invading inflammatory cells, including the oxidative stress caused by liberation of oxygen radicals during the activation of the inflammatory cells;

an antagonist of prostaglandin synthesis; and

an agent that induces tissue regeneration.

Nonlimiting examples of an agent to competitively inhibit alkylation at —SH and >NH sites, e.g., of one or more proteins of interest include taurine, essential amino acid, non-essential amino acid, aromatic amino acid, phenylalanine, tyrosine, tryptophan, sulfur amino acid, cysteine, gluthathione, methionine, homocysteine, urea cycle amino acid, arginine, citrulline, ornithine, glutamate amino acid, glutamic acid, GABA, glutamine, proline, hydroxyproline, aspartic acid, asparagine, threonine amino acid, threonine, glycine, serine, alanine, branched chain amino acid (BCAA), leucine, isoleucine, valine, metabolite amino acids, lysine, carnitine, or histidine. Combinations are possible.

Nonlimiting examples of an agent to cleave (reduce) —SS— to —SH include N-acetyl cysteine, glutathione, ascorbic acid, or cysteine. Combinations are possible.

Nonlimiting examples of an agent to scavenge reactive oxygen species and minimize oxidative stress include hydrophobic oxyradical scavengers, hydrophilic oxyradical scavengers, amphiphilic oxyradical scavengers, alpha-keto-glutarate, alpha-keto-glutarate ethyl ester, alpha-keto-glutarate propyl ester, alpha-tocopherol, beta-tocopherol, gamma-tocopherol, alpha-tocopherol ethyl ester, beta-tocopherol ethyl ester, gamma-tocopherol ethyl ester, alpha-tocopherol propyl ester, beta-tocopherol propyl ester, gamma-tocopherol propyl ester, pyruvate, indole-pyruvate salt, sodium pyruvate, potassium pyruvate, ethyl ester of pyruvate, propyl ester of pyruvate, alpha-keto-butyric acid, or alpha-keto acid. Combinations are possible.

Nonlimiting examples of a substrate to provide metabolic and regenerative support and maintain the status of tissue reduction-oxidation include alpha-keto glutarate, glutarate, fructose, glucose, pyruvate, calcium pantothenate, or sodium pantothenate. Combinations are possible.

Nonlimiting examples of an agent that protects against the diverse effects of invading inflammatory cells, including the oxidative stress caused by liberation of oxygen radicals during the activation of the inflammatory cells include sodium citrate, citric acid, or potassium citrate. Combinations are possible.

Nonlimiting examples of an agent to decrease prostaglandin synthesis include salicylate, acetyl-salicylate, salicylic acid, indomethacin, or dexamethasone. Combinations are possible.

Nonlimiting examples of an agent to aid in tissue regeneration include retinol palmitate, retinol acetate, insulin, NGF (nerve growth factor), EGF (epithelial growth factor), or growth factors, Combinations are possible.

The above components (1-7) are considered to be the active ingredients.

Alpha keto glutarate (AKG) is a precursor to glutamine. Unlike glutamine, the gut does not recognize AKG. Therefore, more AKG passes through the digestive system as compared to free glutamine. Once in the body, AKG can be converted to glutamine as necessary.

Nonlimiting examples of effective amounts of the agent to competitively inhibit alkylation at —SH and >NH sites, e.g., of one or more proteins of interest range from about 10-50% by weight of active ingredients, which range includes 10, 11, 12, 13, 14, 15, 16, 17, 18, 19, 20, 22, 24, 26, 28, 30, 32, 34, 36, 38, 39, 40, 41, 42, 43, 44, 45, 46, 47, 48, 49, and 50% by weight of active ingredients, and any subrange therebetween.

Nonlimiting examples of effective amounts of the agent to cleave (reduce) —SS— to —SH range from 1.5-20% by weight of active ingredients, which range includes 1.5, 1.6, 1.7, 1.8, 1.9, 2, 3, 4, 5, 6, 7, 8, 9, 10, 11, 12, 13, 14, 15, 16, 17, 18, 19, and 20% by weight of active ingredients, and any subrange therebetween.

Nonlimiting examples of effective amounts of the agent to scavenge reactive oxygen species and minimize oxidative stress range from 10-75% by weight of active ingredients, which range includes 10, 11, 12, 13, 14, 15, 16, 17, 18, 19, 20, 22, 24, 26, 28, 30, 35, 40, 45, 50, 55, 60, 70 and 75% by weight of active ingredients, and any subrange therebetween.

Nonlimiting examples of effective amounts of the substrate to provide metabolic and regenerative support and maintain the status of tissue reduction-oxidation range from 2-15% by weight of active ingredients, which range includes 2, 3, 4, 5, 6, 7, 8, 9, 10, 11, 12, 13, 14, and 15% by weight of active ingredients, and any subrange therebetween.

Nonlimiting examples of effective amounts of the agent that protects against the diverse effects of invading inflammatory cells, including the oxidative stress caused by liberation of oxygen radicals during the activation of the inflammatory cells range from 1.5-15% by weight of active ingredients, which range includes 1.5, 1.6, 1.7, 1.8, 1.9, 2, 3, 4, 5, 6, 7, 8, 9, 10, 11, 12, 13, 14, and 15% by weight of active ingredients, and any subrange therebetween.

Nonlimiting examples of effective amounts of the agent to decrease prostaglandin synthesis range from 1-10% by weight of active ingredients, which range includes 1.1, 1.2, 1.3, 1.4, 1.5, 1.6, 1.7, 1.8, 1.9, 2, 3, 4, 5, 6, 7, 8, 9, and 10% by weight of active ingredients, and any subrange therebetween.

Nonlimiting examples of effective amounts of the agent to aid in tissue regeneration range from 1-10% by weight of active ingredients, which range includes 1.1, 1.2, 1.3, 1.4, 1.5, 1.6, 1.7, 1.8, 1.9, 2, 3, 4, 5, 6, 7, 8, 9, and 10% by weight of active ingredients, and any subrange therebetween.

In one embodiment, the composition includes an amount effective for preventing or treating sulfur mustard or Half Mustard toxicity or damage of taurine, N-acetyl cysteine, alpha-keto glutarate, pyruvate, glucose or galactose, insulin, salicylate, citrate, indomethacin or dexamethasone, free or esterified retinol, and free or esterified tocopherol.

In one embodiment, the composition is effective in treating and/or preventing sulfur mustard and Half Mustard-induced damage. One embodiment includes the following compounds, listed below:

Taurine: (Membrane stabilization and inhibition of N alkylation, Anti-oxidant).

N-acetyl cysteine: (Reduction of —S— to 2-SH).

Alpha-keto-glutarate: (Oxyradical scavenger and metabolic support via malate-aspartate shuttle).

Pyruvate: (Oxyradical scavenger and metabolic support via acceleration of glycolysis) (31-34).

Glucose: (Metabolic and bioenergetic support).

Insulin: (Promote glucose availability to cells, stimulates mitosis and tissue regeneration).

Pantothenate: (Provide metabolic support via Coenzyme A).

Salicylate: (Inhibit prostaglandin synthesis).

Citrate: (Modulate invasion by inflammatory cells).

Indomethacin or Dexamethasone (Inhibit prostaglandin synthesis).

Retinol palmitate or other esters of retinol, (epithelial regeneration).

Free or esterified alpha-tocopherol (Prevent oxidation of ointment base and membrane lipids).

The composition may be in the form of a cream or ointment or any other form as appropriate. It may contain one or more pharmaceutically or dermatologically acceptable adjuvants or carriers. Nonlimiting examples of these include petrolatum, lanolin, mineral oil, tween, combinations thereof, or the like. In the case of corneal application, the composition may be isotonic.

The composition may be suitably applied prior to, during, or after exposure. In certain embodiments, the composition is applied to a subject at risk for exposure to sulfur mustard or half mustard gas. In other embodiments, the composition is applied to the affected area after exposure. The composition may be suitably applied to the dermis or conjunctiva or the cornea, or a combination thereof.

Methods of using the compositions of the present invention are contemplated. The compositions are useful for treating a subject at risk for or exposed to sulfur mustard and/or half mustard. As used herein, the terms “treat”, “treating” and/or “treatment” refers to acting upon with a composition of the present invention to improve or alter an outcome; the skilled artisan is aware that the improvement or alteration may be in whole or in part and may not be a complete cure. Treating may also comprise treating a subject at risk for developing a disease and/or condition.

The subject may be a mammal, for example, a human but includes any subject at risk for or exposed to sulfur mustard and/or half mustard gas.

Sulfur mustard is a highly reactive substance, which interacts with a variety of cellular constituents, affecting their structure and function. Mechanistically, one of the first reactions upon contact with the aqueous phase of the tissue environment is that sulfur mustard is hydrolyzed, which leads to the formation of the corresponding thio-diglycol derivative and hydrochloric acid. That this reaction takes place in the body has been proven by the presence of ³⁵S-thiodiglycol in urine following administration of ³⁵S-mustard (4).

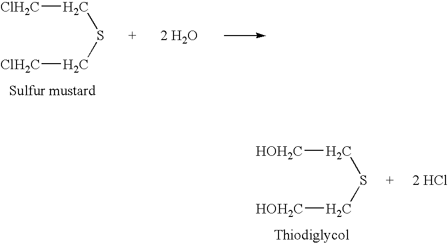

That the hydrochloric acid produced in the above reaction may be the initial irritant is often discounted. However, the toxic effect of a localized change in pH at the site of hydrolysis cannot be entirely ruled out. The toxicological significance of the thio-diglycol generated in the above reaction also remains unexplored, although pharmacologically, such compounds are known to act as CNS depressants.

The above hydrolysis proceeds through the following steps, generating cyclic intermediates containing positively charged sulfur atoms (sulfonium intermediates) (2).

The sulfonium intermediates generated during the above series of hydrolytic reactions act as strong electrophiles. These electrophilic intermediates act as strong alkylating agents, which alkylate various groups such as —NH₂, >NH, >N—, —SH, —S—, —OH, —O—, and the like. This sulfur mustard induced alkylation leads to structural modifications in several soluble as well as structural components of the cells containing the above groups. The toxicity of sulfur mustard has, therefore, been attributed largely to its alkylating activity and consequent modifications in the properties of biological molecules, e.g., proteins, peptides, thiols, nucleic acids, nucleotides and amino acids, and the like (1,5). Hence the pathophysiological effects of the compound can be exerted through modifications and attenuations in the normal functions of several soluble and structural components of the cell, including the cell membranes. Since the cell membrane is the initial site of contact when tissue is exposed to mustard, it is believed that sulfur mustard alkylates the intrinsic membrane proteins at their —NH₂ and —SH residues and consequently damage the membrane transport functions intimately involved in the maintenance of the intracellular composition such as the maintenance of high intracellular ions such as potassium, calcium and other vital components. Although mustard will penetrate the cell and induce alkylation, damage to the outer limiting cell membrane further facilitates intracellular penetration followed by alkylation of structural and soluble components therein. The damage to intracellular structure is evidenced by the breakage of the nuclear membrane that follows mustard exposure. In addition, disintegration of the DNA strands occurs (3), affecting tissue regenerating and repair mechanisms with eventual death due to apoptosis. The latter effects are believed to be similar to the effects of UV and X-ray, wherein DNA damage has been attributed to the attack of free radicals. DNA strand breakage due to mustard is believed to be initiated by alkylation of nucleic acids at purine bases (1,3,6-8). A favorable alkylation site in the DNA strand is the 7th nitrogen of the guanine residue because of the high electron density in the region of —N═CH— bonds. Alkylation of —NH₂ not flanked by additional double bond is comparatively a weaker reaction (1).

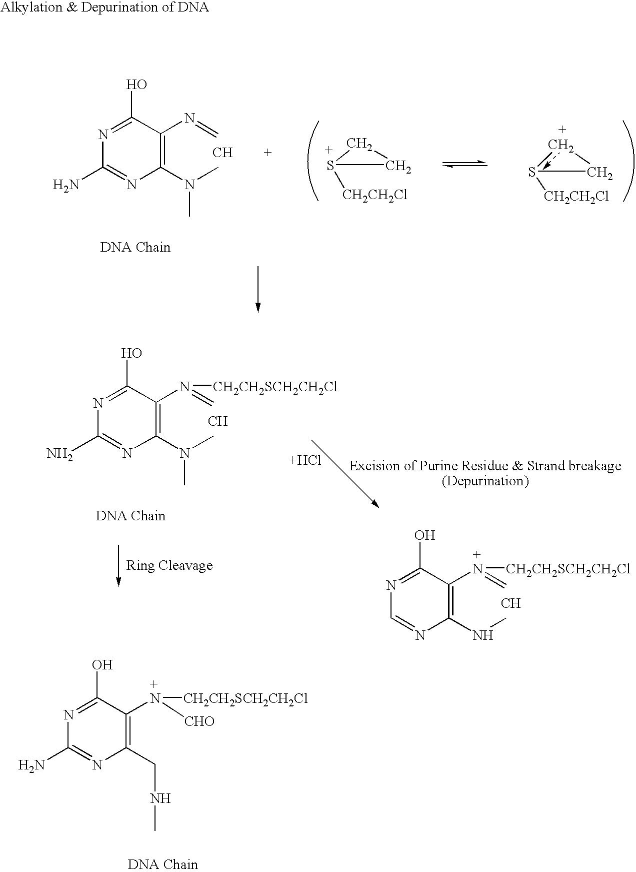

The depurinated nucleic acids are less stable and are hydrolyzed further, either spontaneously or enzymatically, for example, by endonucleases, causing aberrations in replication and transcription mechanisms. The depurination of nucleic acids and/or polynucleotides stimulates enzymatic polymerization of adenosoyl pyrophosphate ribose units (3, 6-8), which are derived from NADase catalyzed hydrolysis of NAD and generates nicotinamide as a co-product.

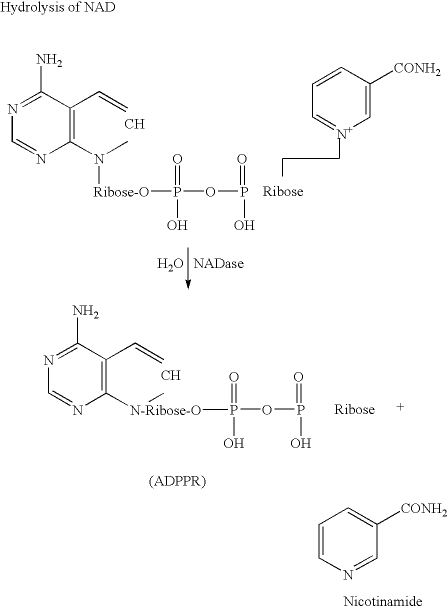

Poly(ADP ribose) produced from ADPPR may combine with histone. During polymerization, as shown below, in poly(ADP ribose) formation, internucleotide linkages are established between C-1 of ribose of the first monomer to C-2 of the subsequent (second) monomer, which is repeated successively. One of the net effects of DNA depurination and activation of NADase and poly(ADP ribose)ₙ polymerase (the enzyme assisting in the polymerization of ADPPR) is the hydrolytic depletion of NAD. Because NAD is an essential pacemaker of intermediary metabolism, its depletion is considered a factor involved in the manifestations of mustard toxicity (8-11). One embodiment of the present invention includes providing metabolic support to enhance the utilization of NAD in tissue glycolysis as a therapeutic route for the prevention and/or treatment of tissue toxicity.

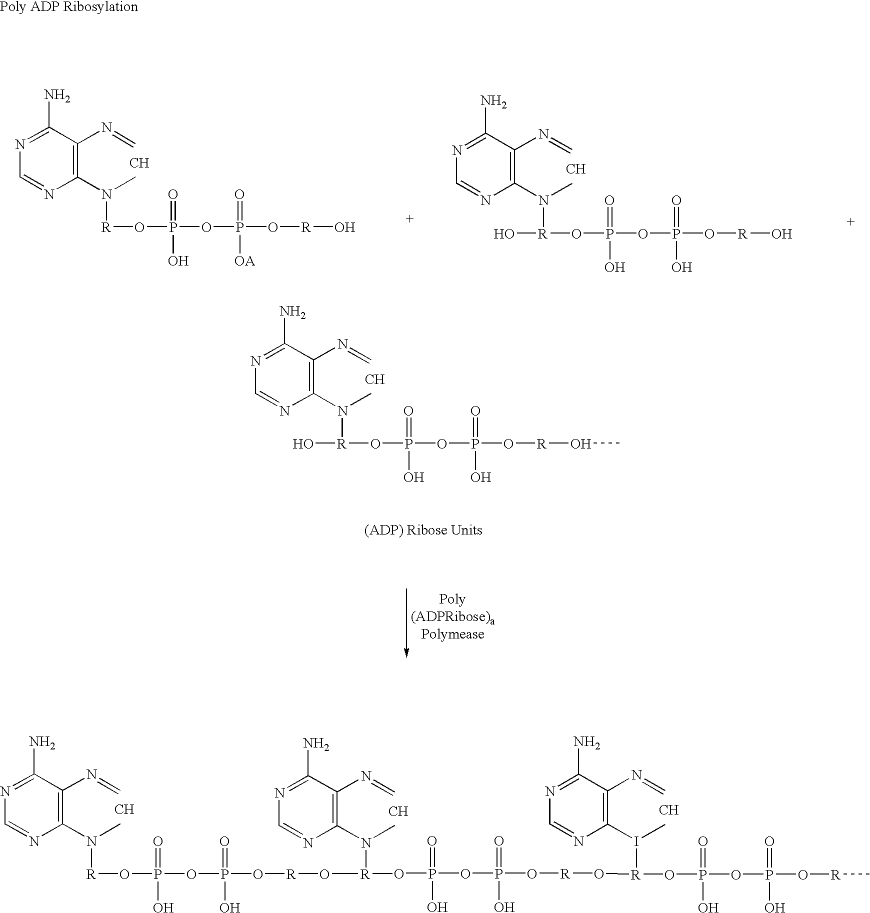

Sulfur mustard toxicity is also attributable to the alkylation of enzymatic sulfylhdryls and consequent metabolic inhibitions (5), resulting in the inhibition of glycolysis as well as respiration in a variety of cells and tissues (minced tumor tissue, normal brain mince, erythrocytes, bone marrow, spleen, thymus and liver (3). Among the specific enzymes, hexokinase and pyruvate kinase have been found to be most adversely affected. Other enzymes such as fumarase, urease, invertase, glycolytic enzymes other than the phosphokinases, proteases, pyruvate oxidase and ascorbate oxidase are reported to be unaffected by sulfur mustard toxicity. However, NAD depletion following exposure to mustard observed in the animal models as well as in the lymphocytes is believed to indicate a more general metabolic effect of this compound. This is indicated further by its limited but significant preventive effect against dermal vesication produced in experimental animals (7,10).

Additional factors such as intracellular accumulation of Ca⁺ (11), loss of cellular adhesion (12,13), and consequent cellular separation and disintegration due to changes in membrane chemistry are however not correctable by NAD supplementation. Specific studies on the mechanism of mustard toxicity and its prevention in the case of skin have so far been very limited and inconclusive (14).

Based in-part on their studies of ocular tissues, the present inventors have found that, apart from its function of transmitting and refracting light into the eye, the structure and physiological function as a barrier against penetration of foreign compounds and particulate material in the inner layers of the cornea are similar to that of the epidermal epithelium. The inventors have found that, like skin, the cornea also functions in maintaining the level of tissue hydration. In the cornea, energy required for maintaining the optimal status of tissue hydration (15-26), ion transport activities and supporting normal regenerative and repair activities, is primarily derived from glycolysis, 65% of glucose metabolized is driven through this process. The remaining portion is utilized via the hexose monophosphate shunt pathway and the citrate cycle (26).

The term “oxidative stress” is generally used to signify the toxic effects of oxygen on structural and metabolic aspects of tissue. Oxygen toxicity results from the obligatory formation of superoxide (I) in most chemical reactions with oxygen, the dismutation of the latter to hydrogen peroxide(II), and the subsequent interaction of the latter with superoxide in the presence of a multivalent metal ion to produce hydroxyl radical via the Haber-Weiss reaction. (27)

| | |
|---|---|
| O₂ + e → O₂^{•-} | (I) Oxygen Reduction |
| O₂ ^{•-} + O₂^{•-} + 2H⁺ → H₂O₂ + O₂ | (II) Dismutation |
| Fe⁺⁺⁺ + O₂^{•-} → Fe⁺⁺ + O₂ | (III) Metal Ion Reduction |
| Fe⁺⁺ + H₂O₂ → Fe⁺⁺⁺ + OH^{•} + OH⁻ | (IV) Fenton's Reaction |
| O₂^{•→} + H₂O₂ → O₂ + OH⁻ + OH^{•} | (V) Haber Weiss Reaction |

The free-electron containing derivatives of oxygen as well as hydrogen peroxide are commonly referred to as “reactive oxygen species”. Normally, about two percent of the respired oxygen is converted to these forms in various tissues of the body. However, inhibition of metabolism due to alkylation and inactivation of proteins (e.g., enzymes), and/or alteration in intracellular ionic composition because of cell membrane damage causes the available oxygen to undergo auto-oxidative reactions instead of being used in the divalent pathways. The auto-oxidative reactions are accompanied with the generation of reactive oxygen species, and the result is direct damage to the cell membranes, intracellular structural elements, enzymes and cofactors involved in tissue metabolism and its overall physiological maintenance.

Normally, defense against the toxic effects of the reactive oxygen species is provided by superoxide dismutase, catalases and glutathione peroxidase, aided secondarily by certain other enzymes and chemical antioxidants. In view of the ineffectiveness of catalases at low physiological peroxide levels, glutathione peroxidase is considered more important in H₂0₂ detoxication (28).

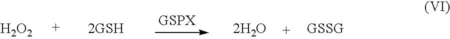

Continued protection, however, requires that the GSSG is rapidly reduced back to GSH. This is primarily accomplished by the utilization of reducing equivalents from NADPH generated in the HMP shunt; the reduction requiring glutathione reductase (GR).

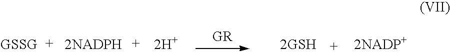

The elimination of peroxide by the above reaction also leads to an indirect elimination of other reactive forms of oxygen. Depletion of GSH from the tissue and hence its unavailability to participate in the antioxidant defenses and the maintenance of various enzymes in the active state such as in the maintenance of glyceraldehyde phosphate dehydrogenase, a key enzyme of glycolysis, aggravates oxidative stress with severe pathophysiological consequences. Since mustard gas is a potent alkylating agent, it is believed that its toxic effect is induced via alkylation of GSH and —SH enzymes. The reactive oxygen species can induce toxicity also by their direct action such as DNA mutation and strand breakage, with consequent aberrations in replication and transcription mechanisms, direct oxidation of enzymatic and structural proteins, amino acids, particularly those containing sulfylhdryl groups (cysteine) and membrane and cytosolic lipid peroxidation. Since the membranes are rich in oxidizable amino acid residues and polyunsaturated fatty acid moieties, membrane damage and consequent loss of the barrier function of the epithelial structures is believed to be the initial malfunctioning factors in the induction of oxidative stress.

The prevailing diversity in the reactivity of mustard at chemical, biochemical, metabolic and structural levels suggest the involvement of multiple factors in its toxicity. In the case of skin, one of its critical functions is to restrict the permeability of unwanted ions, soluble organic toxins and particulate materials such as bacterial invertebrate pathogens. While all the layers of the skin structure are important in the performance of this function, the tight packing of the epithelial cells in the stratum corneum is the most important factor regulating the permeability of the particulate matter. Its action guarding against the penetration of soluble organic compounds and electrolytes is related to the hydrophobicity of the outer cell membrane and the functional orientation of the transport pumps, particularly the Na—K-ATPase and related symports and antiports.

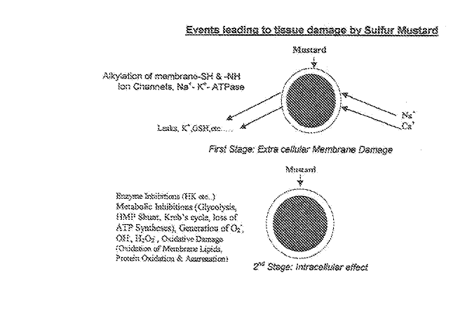

One early event in the mustard toxicity is triggered by its adverse effect on cell membrane permeability (29,30), facilitating further its intracellular penetration and consequent alkylation of various cytoplasmic, mitochondrial and intranuclear enzymatic and nonenzymatic constituents. This has an overall effect of inhibiting cellular metabolism involved in energy production necessary to drive the transport pumps and tissue biosynthetic activities. The metabolic inhibition finally leads to structural damage of the cells due to the diversion of available oxygen from its divalent pathway of reduction with the eventual formation of water, expected to take place during normal metabolism, to the monovalent pathway of its reduction generating highly damaging oxygen radicals, which upsets tissue physiology in a variety of ways such as oxidation of enzyme-SH and inhibition of their activities, lipid peroxidation associated with membrane damage and oxidation of soluble and cytoskeletal proteins etc. FIG. 7 presents a graphical sketch of events leading to tissue damage by sulfur mustard.

Some of the compounds are known to have additional beneficial effects not mentioned herein. Also, they are all endogenous to the body. Hence the likelihood any significant toxicity on external application is highly remote.

### EXAMPLES

The preparation of one embodiment of the composition (named, “VM” or “VM1” herebelow) is described. Since VM is a mixture containing hydrophobic as well as hydrophilic materials, a special procedure was developed to formulate its preparation. Stock solutions were prepared as follows:

Stock solution #1: The following ingredients were added to 100 ml of water at 60 degrees:

| | | |
|---|---|---|
| Taurine | 5 | g |
| Calcium pantothenate | 1.4 | g |
| Sodium pantothenate | 1.4 | g |
| Glucose | 1.2 | g |
| Trisodium citrate | 0.8 | g |
| Disodium EDTA | 0.1 | g |
| Acetyl salicylic acid | 1 | g |
| N-acetyl cysteine | 1 | g |
| Insulin | 10 | mg |
| After cooling the above solution to room | | |
| temperature, the following additions were | | |
| made: | | |
| Sodium pyruvate | 4.5 | g |
| Alpha ketoglutarate | 9.6 | g |

The pH was then adjusted to 6.5 by adding 6 ml of 1N NaOH.

Stock solution # 2 was prepared:

| | | |
|---|---|---|
| Retinol palmitate | 600 | mg |
| Vitamin E acetate/palmitate | 600 | mg |
| Indomethacin | 300 | mg |

Dissolve in 30 ml of heavy mineral oil by warming in boiling water bath.

Stock mixture #3 was prepared:

Lanolin 10 parts

Yellow petrolatum 90 parts.

Preparation of the VM Ointment: in a Blender, Add the Following:

1. Stock mixture #3: 90 g

2. Stock mixture # 2: 20 ml

3. Tween 60: 50 ml

4. Methyl cellulose: 370 mg in 10 ml water

Mix and blend gently.

To the mixture obtained above, add 60 ml of stock solution #1 and blend. Final volume of the mixture: 230 ml

Take out 100 ml and treat it as VM1.

To the rest of the mixture, add 50 mg Dexamethasone in 10 ml of heavy mineral oil.

Overall composition of VM/100 ml of semi-solid material:

| | | |
|---|---|---|
| Lanolin | 3.9 | g |
| Yellow petrolatum | 35.2 | g |
| Retinol palmitate | 0.1739 | g |
| Vitamin E acetate | 0.1739 | g |
| Indomethacin | 0.0869 | g |
| Methyl cellulose (2000cps) | 0.1609 | g |
| Taurine | 1.3 | g |
| Calcium pantothenate | 0.365 | g |
| Sodium pantothenate | 0.365 | g |
| Glucose | 0.313 | g |
| Trisodium citrate | 0.2087 | g |
| Disodium EDTA | 0.026 | g |
| Acetyl salicylic acid | 0.2609 | g |
| N-acetyl cysteine | 0.2609 | g |
| Insulin | 0.0026 | g |
| Sodium pyruvate | 1.17 | g |
| Alpha ketoglutarate | 2.5 | g |

The present inventors have found that application of the above mixture to the CEES exposed skin offers substantial prevention against CEES induced tissue damage as described herein. The similarity in the mechanisms of CEES action and sulfur mustard extrapolates to an expected similar efficacy for sulfur mustard. Thus, the present inventors have developed a composition effective for both prophylactic and post-exposure prevention against tissue damage caused by sulfur mustard.

Mouse Studies: Evidence of the Effectiveness of VM Against Skin Damage Caused by Half Mustard. The effect of VM ointment against the development of necrotic changes on the skin caused by Half Mustard was studied by its application on mouse skin pre-exposed to CEES and determining the attenuation of the pathology with reference to the control group where the animals exposed to CEES were not treated with VM (control). CD-1 mice weighing approximately 25 g were used in these investigations. The animals were first anesthetized by intramuscular injection of a mixture of ketamine and xylazine (66 mg of ketamine and 7 mg of xylazine/kg body weight). Immediately after the onset of anesthesia, a small area on the flank was encircled and clipped to remove hairs. 10 microliters of a freshly prepared solution of CEES in propylene glycol (20 microliter CEES per 100 microliter of PG) was painted on the encircled area covering 1.5 cm². After 10 minutes of this application, the sites were treated with the VM ointment. The treatment was repeated every hour till 3 hours. They were again treated at two hourly intervals till the end of the day. A total of 6 treatments were given on the first day. On subsequent days, four treatments were given at intervals of 1.5 hours. In these particular experiments, treatment was continued for five days. After the treatments, the animals were again anesthetized and skin sites cut out and fixed in 10% buffered formalin. They were then processed for histology.

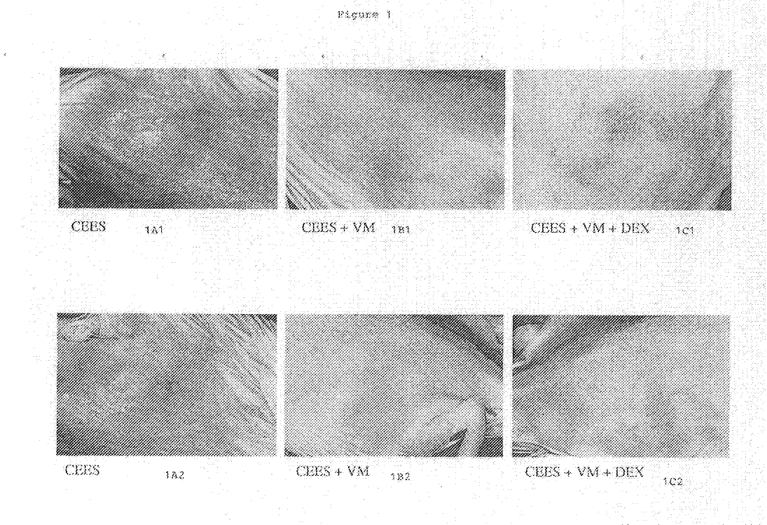

FIGS. 1A₁ and 1A₂ represent the external appearance of the skin after 5 days of CEES application. In the animals where skin was painted with CEES as described above, the region of application appeared necrotic and rougher than normal. Some time the skin would become bare due to total exfoliation. In animals where the CEES application was followed by treatment with VM, development of such degenerative changes were highly attenuated, the appearance in this case being very close to that in the normal animals (FIGS. 1B₁, 1B₂)/Addition of dexamethasone to VM did not result in an increased benefit, (FIG. 1C₁, 1C₂).

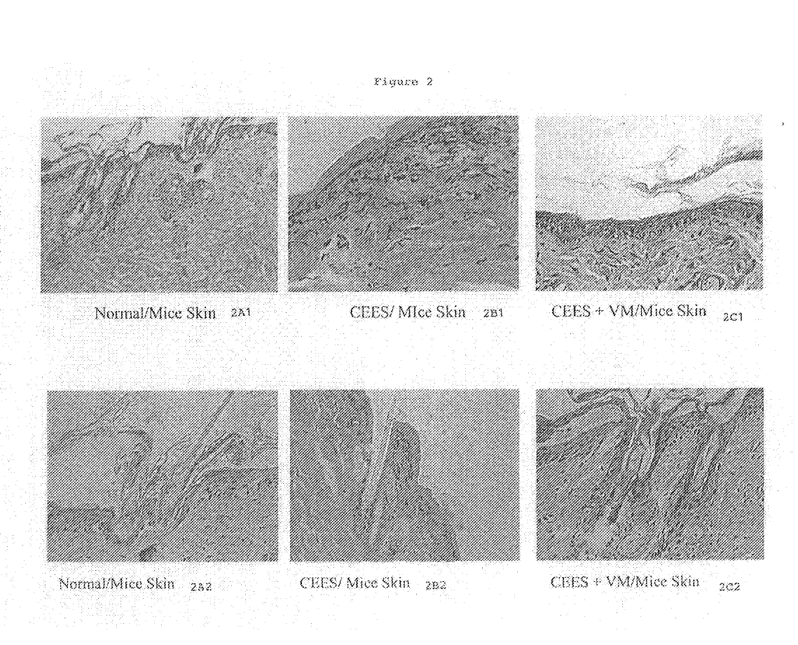

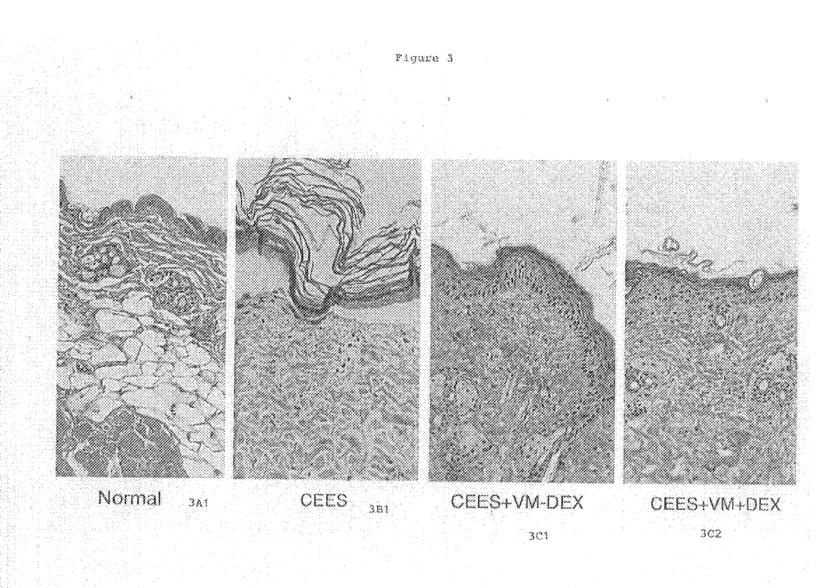

FIGS. 2 to 4 represent the histology of the skin samples in three groups of animals. As apparent in FIG. 2A₁, 2A₂, 3A₁, the epidermis in the normal group is made up of an external layer of stratum corneum consisting of some denuding keratinocytes and free keratin lamellae derived from the dying or dead keratinocytes. This is followed internally by 2 to 3 layers of live keratinocytes in different stages of differentiation. The inner most layer (stratum germinativum) of these epithelial cells rests on a basement membrane, separating the epidermis from dermis. The latter is made up of collagen and elastic fibers and contains, in addition, some fibroblasts and inflammatory cells. The dermis also contains normal glandular structures and hair follicles. As would be apparent from the FIGS. 2B₁ and 2B₂ and 3B₁, on application of CEES, the epidermis gets completely separated from the dermis in certain regions and partially in certain other regions (FIG. 3B₁), giving rise to sub-epithelial space and accumulation of cellular debris and fluids culminating into blister formation. Therefore a structural and physiological damage to the epidermis and blister formation characteristic of HD damage is fully mimicked by Half Mustard.

As apparent from FIGS. 2C₁ & 2C₂ as well as 3C₁ & 3C₂, application of the VM formulation has a significant protective effect against CEES induced damage. The separation of the epithelium caused by CEES is nearly fully prevented. In fact VM seems to maintain the epidermis as well as dermis in much better physiological state as compared to normal, a apparent in FIGS. 2C₁, 2C₂ and 3C₁. Addition of dexmethasone to VM (FIG. 3C₂) was without any additional improvement over 3C₁, where treatment was done with VM without any dexamethasone. The disposition of the epithelium along the hair shaft also remains well maintained. The number of inflammatory cells is also less in this case. As apparent (FIG. 2C₂), application of VM while preventing the tissue against damage by CEES, also minimizes scab formation. This is more apparent from FIGS. 1B₁ and 1B₂. Hence treatment leads to a cosmetically better maintenance of the lesion during healing. This is also likely to minimize unwanted vascularization often noticed in such burns.

Compounds known to inhibit various sulfur mustard based alkylation reactions, and capable of acting as antioxidants (via their ability to scavenge various reactive species of oxygen such as super oxide, hydrogen peroxide and hydroxyl radicals) are effective in preventing CEES induced damage to cornea also. Visual disability, initiated by damage to corneal epithelium is one of the earliest disabling manifestations of exposure to this gas. It is strongly believed that the mechanism of sulfur mustard induced damage in the war field is indeed similar to that of the CEES induced damage to animals observed under the laboratory conditions. Therefore, the present observations on the prevention of Half Mustard damage by the formulation developed is likely to be useful in treatment against tissue damage caused by exposure to the actual mustard (HD) as well.

Rat Model Studies of Sulfur Mustard Toxicity. In order to further affirm the efficacy of the formulation, additional studies have been conducted with rats using the following protocol: Sprague-Dawley rats weighing about 150 g were anesthetized by intra-muscular injection of a mixture of ketamine and xylazine (66 mg ketamine and 7 mg of xylazine/kg body weight). Immediately after the onset of anesthesia, a small area was demarcated on the flank and clipped to remove hairs. 10 ul of a freshly prepared solution of CEES in propylene glycol (20 microliters CEES per 100 microliters of Propylene Glycol) was painted on the circumscribed area of skin covering 1.5 square centimeters. After 6 minutes of this application, the sites were treated with the VM ointment. The treatment was repeated every hour till 3 hours. They were again treated at 2 hourly intervals till the end of the day. A total six treatments were given on the first day. On subsequent days, four treatments were given at intervals of 1.5 hours. Treatment was continued for six days. After the treatments, the animals were again anesthetized and skin sites cut out and preserved in 10% buffered formalin. They were then processed for histology.

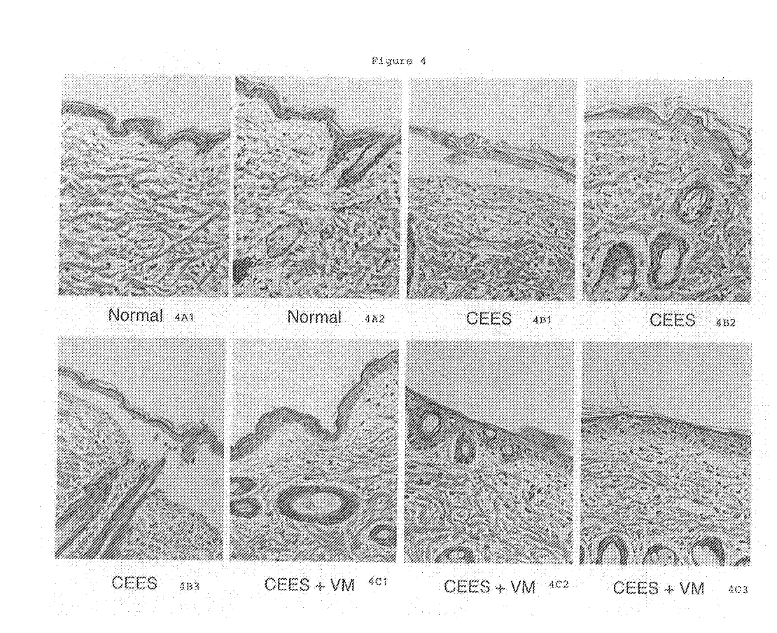

The histologies of the representative skin samples obtained form normal, CEES treated and CEES plus VM treated groups are presented in FIGS. 4A to 4D. FIGS. 4A₁ and 4A₂, show the histological structure of the normal rat skin. The structure represents typical thin skin architecture, similar to that in mouse; the epidermis consisting of one or two layers of epithelial cells covered by stratum corneum made up of flattened enucleated keratinocytes. The dermis, as expected, consists of loosely arranged elastin and collagen fibers, fibroblasts, some macrophages, glandular structures and embedded hair follicles (FIGS. 4A₁ & 4A₂).

One of the most significant and striking effects of CEES application, as observed in experiments with mice, is the separation of the entire epidermis from the underlying dermis. The space in between is filled with fluid and some cellular debris, characteristic of blister formation. (FIGS. 4B₁, 4B₂, and 4B₃). In some places, the entire epidermis becomes exfoliated, leaving the dermis bare. Also, the dermis becomes enriched and infiltrated with additional amounts of inflammatory cells and debris. As observed in experiments with mouse, application of VM, as described above, prevented most of the CEES induced structural alterations, (FIG. 4C₁, 4C₂ & 4C₃). The epithelium remained intact, remaining fully adherent to the dermis along the basement membrane. Blister formation was completely prevented. The glandular structure in the dermis also appeared healthy. It is also interesting to note that the epithelial layer, especially in the glandular areas, appeared hyperplastic and maturing faster, which may represent an injury response.

Beneficial Effect of VM as Evident by its Preventive Effect against Apoptosis. Skin epithelial cells are programmed to differentiate and migrate in layers towards the exterior. With this migration, the cells moving outward become becoming less active and keratinized and are finally sloughed off as dead cells. A layer of geminative epithelium however remains attached to the basement membrane. These cells continue to divide by mitosis, providing additional layers of cells for continued replacement of the pre-existing layers that continuously move forward for eventual denudation. Since adequate maintenance of cellular metabolism is crucial for cellular viability, the present inventors hypothesized that application of VM may also to inhibit the acceleration of the denudation process and exposure of the bare fascia and the dermis resembling ulcers encountered in mustard burns. The exfoliative process indeed is representative of apoptosis characteristic of tissues undergoing continued replacement. The overall preventive effect of VM as described above was hence conceived to be due to its effect against DNA degradation and consequent apoptosis because of its oxyradical scavenging and consequent anti-oxidative effects, the generation of oxyradicals being attributable to the inhibition of cellular metabolism and consequent diversion of the molecular oxygen to the monovalent reductive pathway.

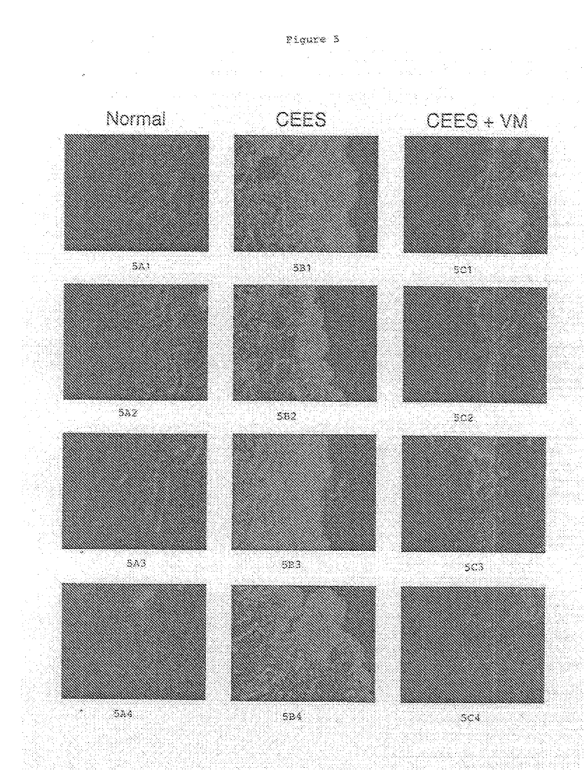

The assessment of apoptosis was done by histochemical detection of DNA fragmentation using TUNEL (35) and Hoechst (bis-benzimide) (36) staining. FIG. 5 shows antero-posterior sections of the flank skin processed through the staining procedure as follows: Freshly isolated lesions were fixed in buffered formalin for a period of 24 hours. The samples were then dehydrated and delipidated by passing through graded concentrations of alcohol and xylene. The samples were then embedded in paraffin for section cutting. The cut sections were mounted on the slides after removing excess of paraffin in hot water and fixed on the slide by gentle warming. Excessive paraffin was further removed by rinsing the sections with xylene. Excessive xylene was then removed from the sections by alcohol treatment. The sections were then hydrated with the staining buffer. The buffered sections were then incubated with proteinase K (20 μg/ml in 10 mM Tris HCL, pH 7.8) for 30 minutes at 37° C. The hydrolyzed proteins and the components of the enzymatic reagents were washed out with phosphate buffered saline. The sections were then incubated for 1 hour with 50 microliters of TUNEL reaction mixture containing terminal deoxynucleotidyl transferase (TdT) and fluorescein-labeled dUTPs. The sections were then rinsed again with PBS, dried at room temperature and examined for detection of nuclear fluorescence at excitation/emission in the range of 450-500/515-565 nm. Photographs were then taken with IP lab software.

Subsequently, the sections were exposed to 100 μl of the reagent containing 1 μM Hoechst reagent (Sigma, St. Louis. MO, Catalogue no. 33258) for 1 hour at 37° C. The slides were then rinsed with PBS and examined for fluorescence at 360/460 nm.

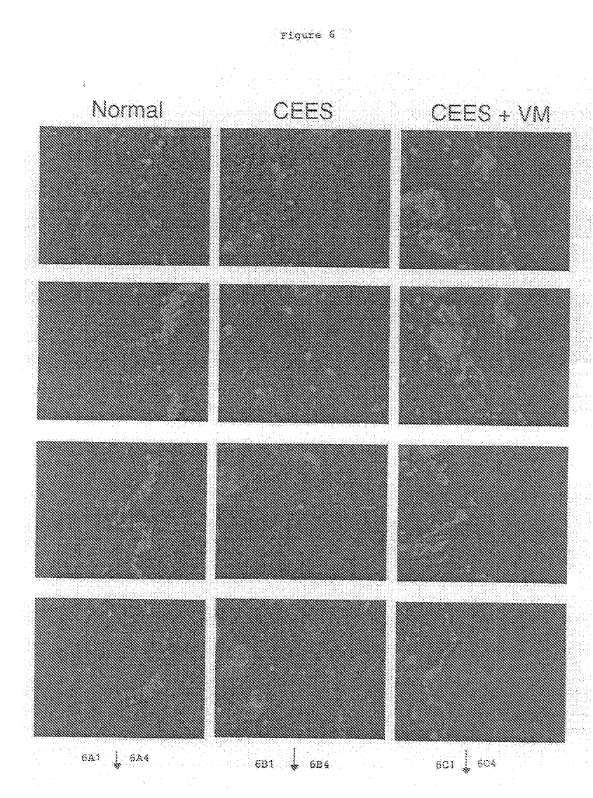

As shown in FIGS. 5A, & A₂, 5A₃ and A₄, reactivity with the TUNEL reagent in the normal specimens was minimal and noticeable very sparingly only in the superficial layer of the epidermis. On the contrary, the cells in the germinal as well as post-germinal layers are highly fluorescent in the sections prepared from the skin after 3 days of CEES exposure (FIG. 5B₁ to B₄). Treatment with VM was highly effective in inhibiting this apoptotic manifestation, the fluorescence in this case (FIG. 5C₁ to C₄) being close to that in the normals. The breakdown of DNA was apparently diminished strongly by VM treatment, as compared to the skin samples of rats not given any VM treatment after CEES application. This inhibition appears related to the oxyradical scavenging compounds present in the VM formulation. The status of the nuclei in cells in the epidermis of the normal skin as apparent by Hoechst stain, is shown in FIG. 6. As clear, the nuclei are well defined, and as expected, are restricted mostly to the epidermis (6A₁ to 6A₄) with smaller numbers in the dermis. In the samples of CEES treated animals, the number of nuclei was found to be substantially decreased (6B₁ to 6B₄). In addition, they were irregular in shape and size. On the contrary, in the CEES+ VM samples, the nuclei are still intact and well demarcated (6C₁ to 6C₄). The nuclei were also normal in the dermis. VM has therefore been found to be highly effective in preventing overall cell death mechanism, as apparent by its inhibitory effect against apoptosis. This inhibitory effect conforms well to the overall normal appearance of the skin superficially as well as the maintenance of its cellular structure apparent histological.

The relevant contents of each of the following (1-36) are hereby incorporated by reference, for all purposes.
1. Gilman A. and Philips F. S. (1946). The biological actions and therapeutic applications of beta-Cloroethyl amines and sulfides. Science 103, 409-415.
2. Lawley P. D. and Brooks P. (1965). Molecular mechanism of the cytotoxic action of difunctional alkylating agents and of resistance to this action. Nature 206, 480-482.
3. Wheeler G. P. (1962). Studies related to the mechanism of actions of cytotoxic alkylating agents: a review. Cancer Res. 22, 651-687.
4. Davison C., Rosman R. S., Bliss L. and Smith P. K (1957). Studies on the metabolic fate of bis(2-Chloroethyl) sulfide (mustard gas) in the mouse and human. Proc. Am. Assoc. Cancer Res. 2, 195.
4. Dixon M. and Needham D. M. (1946). Biochemical research in chemical warfare agents. Nature 158, 432-438.
6. Hayaishi L. and Ueda K. (1982). ADP ribosylation Reactions in Biology and Medicine. Academic Press New York.
7. Papirmeister B., Gross C. L., Meir H. L., Petrali J. P and Johnson J. B. (1985) molecular basis of mustard-induced vesication. Fundamentals and Applied Toxicology 5, S134-S149.
8. Papirmeister B., Westling A. W. and Schroder J. (1969). Relevance of DNA damage to the vesicant action of sulfur mustard. Edgewood Arsenal Tech. Publications #4294.
9. Marlow D. D., Mershon M. M., Mitcheltree L. W., Petrali J. P and Jaax G. P. (1090) Sulfur mustard induced toxicity in hairless guinea pigs. J. Toxicol & Ocular Toxicol 9, 179-192.
10. Yourick J. J., Dawson J. S, and Mitcheltree L. W. (1992). Sulfur mustard-induced microvesication in hairless guinea pigs: Effect of short-term niacinamide administration. Toxicol. & Appl. Pharmacol. 117, 104-109.
11. Petrali J. P., Hamilton T. A., Mills K. R. and Day, R. (1993). Cell injury and calcium accumulation following sulfur mustard exposure. Proc. 51st Ann. Meeting of Microscopy, Society of America, San Francisco Press, p. 322-323.
12. Herrman H. and Hickman F. H. (1948). Loosening of the corneal epithelium after exposure to mustard. Bull. of the Johns Hopkins Hospital. 82, 213-224.
13. Herrman H. and Hickman F. H. (1948). The adhesion of epithelium to stroma in cornea. Bull. Johns Hopkins Hospital, 82, 2873.
14. Friedenwald J. S, and Woods A. C. (1948). Studies on the physiology, biochemistry and cytopathology of the cornea in relation to injury by mustard gas and allied toxic agents. 82, 326-337.
15. Herrman H. and Hickman F. H. (1948). Exploratory studies on corneal metabolism. Bulletin Johns Hopkins Hospital 82, 225-250.
16. Herman H. and Hickman F. H. (1948). Further experiments on corneal metabolism in respect to glucose and lactic acid. Bull. Johns Hopkins Hospital 82, 260-272.
17. DeRoth A. (1950). Respiration of the cornea. Archives of Ophthalmology 44, 666-676.
18. DeRoth A. (1951). Glycolytic activity of cornea. Arch. Ophthal. N.Y. 45, 139-148.
19. Langham M. L (1954). Glycolysis in cornea. J. Physiol. 126, 396-403.
20. Riley M. and Winkler B. (1990). Strong Pasteur effect in rabbit corneal epithelium preserves fluid transport under anaerobic conditions. J. Physiol. 426, 81-93.
21. Robbie W. A., Leinfelder P. J. and Duane T. D. (1947). Cyanide inhibition of corneal respiration. Am. J. Opthalmol. 30, 1381-1386.
22. Riley M. V. (1988). Oxygen Requirements of the Cornea: In “The Cornea: Transactions of the World Congress on the Cornea III”. Ed. H. Dwight Cavanaugh. Raven Press Ltd. NY, p. 207-209.
23. Herrman H. and Hickman F. H. (1948). The utilization of ribose and other pentoses by the cornea. Bulletin Johns Hopkins Hospital. 82, 287-294.
24. Kinoshita J. H. (1957). Stimulation of the phosphogluconate oxidation pathway by pyruvate in bound corneal epithelium. J. Biol. Chem. 228, 247-253.
25. Kinoshita J. H. and Masurat T. (1954). The direct oxidative carbohydrate cycle of bovine corneal epithelium. Arch. Biochem. Biophys 53, 9-19.
26. Kinoshita J. H. and Masurat T. (1959). Aerobic pathway of glucose metabolism in bovine corneal metabolism. Am. J. Opthalmol. 48, 47-52.
27. Haber F. and Weiss J. (1934). The catalytic decomposition of hydrogen peroxide by iron salts. Proc. Royal Soc. (London) Series A. 147, 333-351.
28. Cohen G. and Hochstein P. (1963). Glutathione peroxidase: The primary agent for the elimination of hydrogen peroxide in the erythrocytes. Biochemistry 2, 1420-1428.
29. Varma S. D., Devamanoharan P. S., Ali A. H., Brozetti J., Petrali J., Lehnert E. and Weir A. (1998). Half Mustard induced damage to rabbit cornea; attenuating effect of Taurine-Pyruvate-Alpha-ketoglutarate-Pantothenate Mixture. J. Ocular Pharmacol. & Therap. 24, 423-428.
30. Varma S. D. Devamonobaran P. S., Ali A. H., Henein M., Petrali J., Brozetti J. and Lehnert E. (1998). Corneal damage by Half Mustard. In vitro preventive studies; A histological and electron microscopic evaluation. J. Ocular Pharmacol. & Therap. 14, 413-421.
31. Varma S. D. and Morris S. M. (1988). Peroxide damage to eye lens in vitro. Prevention by pyruvate. Free Rad. Res. Comm. 4, 283-290.
32. Varma, S. D. and Richards, R. D. (1988). Visual disorders and oxidative reactions. In “Cellular Antioxidant Defense Mechanisms”, CRC Press In, 71-83.
33. Varma S. D., Devamanoharan P. S, and Morris S. M. (1990). Photoinduction of cataracts in rat lens in vitro. Preventive effect of pyruvate. Exp. Eye Res. 50, 805-812.
34. Henein M., Devamanoharan P. S, and Varma S. D. (1991). Prevention of galactose cataract by pyruvate. Lens & Eye Tox. Res. 9, 25-26.
35. Hegde K R, Varna S D. Morphogenetic and apoptotic changes in diabetic cataract. Prevention by pyruvate. Mol Cell Biochem, 2004.
36. Hegde K R, Varma S D. Cataract in experimentally diabetic mouse: Morphological and apoptotic changes. Diabetes Obes. Motab, 2004.

1. A composition, comprising, in amounts effective to treat or prevent sulfur mustard or half sulfur mustard induced toxicity or skin damage:
an agent that inhibits alkylation of —SH and >NH protein groups;
an agent that reduces —SS— to —SH;
a scavenger of reactive oxygen species;
a substrate that maintains tissue reduction-oxidation status;
an agent that protects against invading inflammatory cells and associated oxidative stress;
an antagonist of prostaglandin synthesis; and
an agent that induces tissue regeneration.

2. The composition of claim 1, wherein the agent that inhibits alkylation of —SH and >NH protein groups is selected from the group consisting of taurine, essential amino acid, non-essential amino acid, aromatic amino acid, phenylalanine, tyrosine, tryptophan, sulfur amino acid, cysteine, gluthathione, methionine, homocysteine, urea cycle amino acid, arginine, citrulline, ornithine, glutamate amino acid, glutamic acid, GABA, glutamine, proline, hydroxyproline, aspartic acid, asparagine, threonine amino acid, threonine, glycine, serine, alanine, branched chain amino acid (BCAA), leucine, isoleucine, valine, metabolite amino acid, lysine, carnitine, histidine, and a combination thereof.

3. The composition of claim 1, wherein the agent that reduces —SS— to —SH is selected from the group consisting of N-acetyl cysteine, glutathione, ascorbic acid, cysteine, and a combination thereof.

4. The composition of claim 1, wherein the scavenger of reactive oxygen species is selected from the group consisting of oxyradical scavenger, hydrophilic oxyradical scavenger, amphiphilic oxyradical scavenger, alpha-keto-glutarate, alpha-keto-glutarate ethyl ester, alpha-keto-glutarate propyl ester, alpha-tocopherol, beta-tocopherol, gamma-tocopherol, alpha-tocopherol ethyl ester, beta-tocopherol ethyl ester, gamma-tocopherol ethyl ester, alpha-tocopherol propyl ester, beta-tocopherol propyl ester, gamma-tocopherol propyl ester, pyruvate, indole-pyruvate salt, sodium pyruvate, potassium pyruvate, ethyl ester of pyruvate, propyl ester of pyruvate, alpha-keto-butyric acid, alpha-keto acid, and a combination thereof.

5. The composition of claim 1, wherein the substrate that maintains tissue reduction-oxidation status is selected from the group consisting of alpha-keto glutarate, glutarate, fructose, glucose, pyruvate, calcium pantothenate, sodium pantothenate, and a combination thereof.

6. The composition of claim 1, wherein the agent that protects against invading inflammatory cells and associated oxidative stress is selected from the group consisting of sodium citrate, citric acid, potassium citrate, and a combination thereof.

7. The composition of claim 1, wherein the antagonist of prostaglandin synthesis is selected from the group consisting of salicylate, acetyl-salicylate, salicylic acid, indomethacin, dexamethasone, and a combination thereof.

8. The composition of claim 1, wherein the agent that induces tissue regeneration is selected from the group consisting of retinol palmitate, retinol acetate, insulin, NGF, EGF, growth factor, and a combination thereof.

9. The composition of claim 1, wherein the agent that inhibits alkylation of —SH and >NH protein groups is present in an amount ranging from 10-50% by weight of active ingredients.

10. The composition of claim 1, wherein the agent that reduces —SS— to —SH is present in an amount ranging from 1.5-20% by weight of active ingredients.

11. The composition of claim 1, wherein the scavenger of reactive oxygen species is present in an amount ranging from 10-75% by weight of active ingredients.

12. The composition of claim 1, wherein the substrate that maintains tissue reduction-oxidation status is preserit in an amount ranging from 2-15% by weight of active ingredients.

13. The composition of claim 1, wherein the agent that protects against invading inflammatory cells and associated oxidative stress is present in an amount ranging from 1.5-15% by weight of active ingredients.

14. The composition of claim 1, wherein the antagonist of prostaglandin synthesis is present in an amount ranging from 1-10% by weight of active ingredients.

15. The composition of claim 1, wherein the agent that induces tissue regeneration is present in an amount ranging from 1-10% by weight of active ingredients.

16. A method for treating sulfur mustard or half sulfur mustard induced toxicity or skin damage, comprising administering to a subject in need thereof a composition, said composition comprising, in amounts effective to treat sulfur mustard or half sulfur mustard induced toxicity or skin damage:
an agent that inhibits alkylation of —SH and >NH protein groups;
an agent that reduces —SS— to —SH;
a scavenger of reactive oxygen species;
a substrate that maintains tissue reduction-oxidation status;
an agent that protects against invading inflammatory cells and associated oxidative stress;
an antagonist of prostaglandin synthesis; and
an agent that induces tissue regeneration.

17. The method of claim 16, wherein the subject is a human.

18. The method of claim 16, wherein the subject has been exposed to sulfur mustard or half sulfur mustard.
